(19) United States
(12) Patent Application Publication
(10) Pub. No.: US 2009/0157179 A1
(43) Pub. Date: Jun. 18, 2009

(54) Ophthalmic Lenses Providing an Extended Depth of Field

(76) Inventors: Candido D. Pinto, Penfield, NY (US); Amanda C. Kingston, Rochester, NY (US); David C. Compertore, Penfield, NY (US); Joel Pynson, Toulouse (FR); Ian G. Cox, Honeoye Falls, NY (US)

Correspondence Address: Bausch & Lomb Incorporated, One Bausch & Lomb Place, Rochester, NY, 14604-2701, US

(21) Appl. No.: 12/332,526

(22) Filed: Dec. 11, 2008

Related U.S. Application Data
(60) Provisional application No. 61/012,867, filed on Dec. 11, 2007.

(51) Int. Cl. A61F 2/16

(52) U.S. Cl. 623/6.11

(57) ABSTRACT

An aspect of the invention is directed to an ophthalmic lens, comprising at least one optic comprising a lens zone. The zone is configured such that, when the lens is located in an average eye, for all object locations along an axis in a range from infinity to 1.0 diopters, for light having a wavelength 550 nm, the wavefront at the retina formed by the lens zone has the following characteristics

$$0.01 \leq \vert \frac{{Z}_{40}}{{Z}_{20}} \vert \leq 5.0 , and$$

0.01≦|Z₆₀|≦1.0 waves of 550 nm light. In some embodiments, 0.01≦|Z₈₀|≦0.5 wave for light having a wavelength 550 nm.

### CROSS-REFERENCE

This application claims the benefit of U.S. Provisional Patent Application No. 61/012,867 filed Dec. 11, 2007 which is incorporated by reference herein.

### FIELD OF INVENTION

The present invention relates to ophthalmic methods and procedures, and more particularly to methods and procedures for providing eye optical systems with extended depths of field.

### BACKGROUND OF THE INVENTION

The use of intraocular lenses (IOLs) to replace natural crystalline lenses of patients is known. A well known drawback of conventional IOLs is that they have a fixed focus and provide a patient with a limited depth of field around a fixed object plane.

Several techniques have been developed or discussed to provide IOLs with extended depths of field. Examples of IOLs using such techniques include accommodative IOLs (i.e., IOLs capable of accommodative movement), multifocal IOLs, pairing of IOLs using a monovision techniques, and IOLs having selected amounts of primary spherical aberration.

Accommodative IOLs (AIOLs) are IOLs that have variable focal planes, the variations achieved by movement of the IOL that occurs in response to an accommodative stimulus of a wearer's eye. For example, some AIOLs rely on contraction and relaxation of the capsular bag under forces from the ciliary muscle to shift the focal plane by movement of the lens. The movement may cause a translation of the lens or change in shape of a surface of the lens.

Multifocal IOLs are IOLs having two or more zones on a lens, each zone having a different focal length. By focusing portions of the light incident on the various zones, the patient's retina is provided with light focused at different focal planes.

Monovision is a technique which involves placing IOLs in each of a patient's eyes, the IOLs having different focal lengths. Accordingly, the patient has relatively near vision for one eye and relatively distant vision in the other eye.

Some have attempted to select amounts of primary spherical aberration to cause light from the IOL to focus at different locations along the IOL's optical axis.

Each of the above techniques has been found to have limitations.

### SUMMARY

Aspects of the present invention are directed to controlling multiple orders of spherical aberration for light along an axis (e.g., the optical axis) of a lens, to provide an extended depth of field to a wearer.

An aspect of the invention is directed to an ophthalmic lens in an average eye, comprising at least one optic comprising a lens zone. The zone is configured such that, when the lens is located in an average eye, for all object locations along an axis in a range from infinity to 1.0 diopters, for light having a wavelength 550 nm, the wavefront at the retina formed by the lens zone has the following characteristics

$$0.01 \leq \vert \frac{{Z}_{40}}{{Z}_{20}} \vert \leq 5.0 , and$$

0.01≦|Z₆₀|≦1.0 waves of 550 nm light.

In some embodiments, the wavefront at the retina formed by the zone has 0.001≦|Z₈₀|≦0.1 waves for light having a wavelength 550 nm. It is to be understood that the indicated amounts of

$$\frac{{Z}_{40}}{{Z}_{20}} ,$$

Z₆₀, and Z₈₀ are measured at the retinal plane.

An aspect of the invention is directed to an ophthalmic lens, comprising at least one optic comprising a lens zone. The zone is configured such that, when the lens is located in an average eye, for all object locations along an axis in a range from infinity to 1.0 diopters, for light having a wavelength 550 nm, the wavefront at the retina formed by the lens zone has the following characteristics

$$0.01 \leq \vert \frac{{Z}_{40}}{{Z}_{20}} \vert \leq 5.0 .$$

In some embodiments an amount of |Z₆₀| and/or |Z₈₀| as described herein may be added.

As is commonly understood, Zemike polynomials (Z_{n, m}), as used in the above equations, can be used to describe a wavefront that is output from a lens using the following equation.

$$W ( ρ , θ ) ≡ {\sum }_{n , m} {Z}_{n , \pm m} {ZP}_{n}^{\pm m} ( ρ , θ ) )$$

Zernike polynomials as used herein are given their conventional definitions, e.g., as described in the Optical Society of America Taskforce for Standardization proposal of Aberration Reporting Schemes of the Human Eye (2000).

ZP₂₀−\sqrt{3}(2p²−1) which is proportional to defocus and piston;

ZP₄₀=\sqrt{5}(6p²−6p²+1) which is proportional to 1^{st} order spherical aberration (SA), defocus and piston;

ZP₆₀=\sqrt{7}(20p⁶−30p⁴+12p²−1) which is proportional to 2^{nd} order SA, 1^{st} order SA, defocus and piston

ZP₈₀=\sqrt{9}(70p⁶−140p⁶+90p⁴−20p²+1) which is proportional to 3^{rd} order SA, 2^{nd} order SA, 1^{st} order SA, defocus and piston

The term “ophthalmic lens” refers to an artificial lens. The term “ophthalmic lens” includes but is not limited to intraocular lenses (IOLs), contact lenses, spectacles, and corneal onlays or inlays.

The unit “waves,” as used herein to specify an amount of spherical aberration, means a length equal to a multiple of a selected wavelength.

A prescription defining an “average eye” as the term is used herein, and which is to be used to determine performance of lenses as described herein, is provided in Table 1. It will be recognized that the prescription is an eye model according to the Liou-Brennan eye model of 1997. Parameters, such as the indexes of refraction of the various media of the model, which are not specified herein have values as specified in the above-specified eye model.

TABLE 1

| Name of | Radius of | | Thickness | |
|---|---|---|---|---|
| Surface | Curv. (mm) | Conic | (mm) | Medium |
| Anterior | 7.770000 | −0.180000 | 0.500000 | Cornea |
| Cornea | | | | |
| Posterior | 6.400000 | −0.600000 | 3.160000 | Aqueous |
| Cornea | | | | Humor |
| Pupil | Infinity | 0.000000 | 0.000000 | Aqueous |
| | | | | Humor |
| Anterior | 12.400000 | −0.940000 | 1.590000 | Anterior |
| Lens | | | | Crystalline |
| Intermediate | Infinity | 0.000000 | 2.430000 | Posterior |
| Lens | | | | Crystalline |
| Posterior | −8.100000 | 0.960000 | 16.238830 | Vitreous |
| Lens | | | | Humor |
| Retina | −13.400000 | 0.000000 | — | — |

It will be appreciated that design and/or performance determination of contact lenses can be achieved with the lens applied to an eye so as to have zero separation from the corneal surface. It will also be appreciated that design and/or performance determination of an intraocular lens (IOL) to be placed in the capsule bag of the lens can be achieved with the IOL being applied so as to be disposed between the anterior and posterior crystalline lens surfaces (and the optical power of said anterior and posterior surfaces removed). The IOL may be located with the anterior surface of the IOL midway between the anterior and posterior crystalline lens surfaces or at another suitable distance (e.g., as determined by the A-constant of the lens). The above lens locations are described by way of example. Any other lens can be applied at any suitable location in the eye model (e.g., the eye sulcus or the anterior chamber). The diameter of the iris in the average eye is not specified by the average eye model, but may be separately specified.

As one of ordinary skill in the art would understand, to determine performance of a lens of a particular dioptric power (or to design a lens of a particular dioptric power) the model eye should be adjusted (by adjusting the location of the retina) such that a best-focus image is obtained on the retina of the model eye for an object at infinity. Although multiple, substantially equivalent techniques may be used to achieve best focus on the retina, for purposes of aspects of this invention, the position of the retina can be adjusted (i.e., by changing the depth of the vitreous humor as specified by the distance between the posterior lens surface and the retina) to achieve best focus on the retina.

Techniques according to aspects of the present invention are applied to a single lens zone; however, a lens in which such a zone resides may be monozonal or multizonal. The presence of two zones in a lens can be recognized by a presence of at least two local maxima in a plot of optical response as a function of vergence. An example of an optical response plot of a two-zone lens in an eye is shown in FIG. 2, where modulation (for a given spatial frequency) as a function of vergence (in diopters) is illustrated. Two local maxima 2a and 2b are shown, one corresponding to vision at an infinite distance and another corresponding to near distance. It will be appreciated that performance of a single zone in a multizonal lens is determined by masking a portion the lens such that only a single zone is present. A boundary between zones can be identified by a discontinuity in a lens surface or a discontinuity in a wavefront that is output from the lens.

To plot modulation as a function of vergence, a modulation frequency of visual significance is selected (e.g., modulation amplitude for a spatial frequency of 100 lp/mm). Although a plot of modulation is illustrated, other suitable optical response parameters may be used such as contrast, strehl ratio or resolution. The wavelength of the light used may be any visually significant bandwidth or wavelength of light, such as the photopic spectrum or the center of the photopic spectrum (e.g., approximately 550 nm). It will be appreciated that lenses according to aspects of the present invention can comprise three or more zones. The peaks are typically separated by at least 0.5 diopters; and in some embodiments the separation is at least 1.0 diopters. It will be appreciated that the locations of the peaks can be used to calculate focal lengths of the various zones.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1A is a plan view of an example of an ophthalmic lens according to aspects of the present invention;

FIG. 1B is a cross sectional side view of the ophthalmic lens shown in FIG. 1A;

FIG. 2 is an optical response plot of a lens having two zones; and

FIG. 3 is a flow chart illustrating a method according to an aspect of the invention.

### DETAILED DESCRIPTION

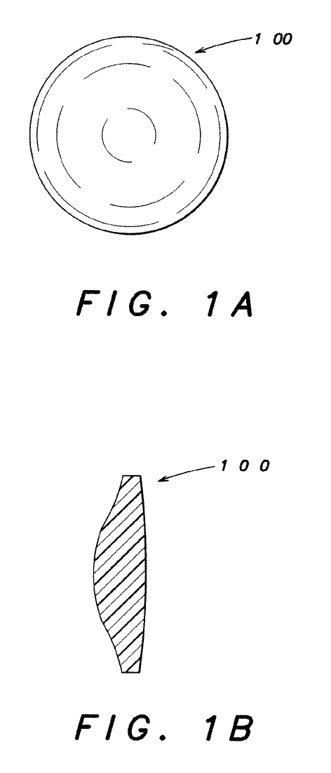

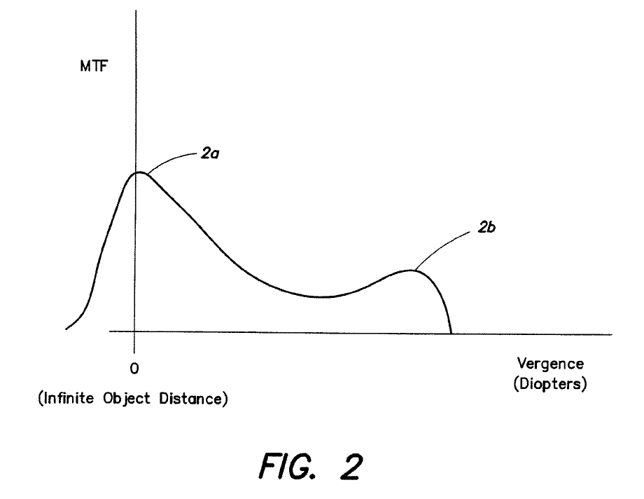

FIG. 1A is a plan view of an example of an ophthalmic lens 100 according to aspects of the present invention. It will be appreciated that non-optical components of the lens may be added in some embodiments (e.g., in intraocular lenses, haptics may be added). FIG. 1B is a cross sectional side view of ophthalmic lens 100. Lenses according to aspects of the present invention can comprise combinations of surfaces having any suitable shape (plano, convex, concave). The illustrated embodiments of lenses have only one zone; however, other embodiments may have multiple zones.

As discussed above, an aspect of the invention is directed to an ophthalmic lens, comprising at least one optic comprising a lens zone. The zone is configured such that, when the lens is located in an average eye, for all object locations along an axis in a range from infinity to 1.0 diopters, for light having a wavelength 550 nm, the wavefront at the retina formed by the lens zone has the following characteristics

$$0.01 \leq \vert \frac{{Z}_{40}}{{Z}_{20}} \vert \leq 5.0 , and$$

0.01≦|Z₆₀|≦1.0 waves of 550 nm light.

The term

$$“ \vert \frac{{Z}_{40}}{{Z}_{20}} \vert ”$$

indicates an absolute value of a ratio of the first order (i.e., primary) spherical aberration (SA) (Z₄₀) and defocus (Z₂₀), corresponding to a distribution of light energy along an axis. Such a ratio describes (and the presence of such aberration operates to provide) a distribution of light energy along an axis of the lens (e.g., the optical axis of the lens). The presence of such an aberration facilitates achievement of an enhanced depth of field. It is to be understood that the selected amounts of

$$\vert \frac{{Z}_{40}}{{Z}_{20}} \vert$$

and |Z₆₀| in a given lens design are selected in conjunction and that, typically a larger amount of one of said quantities may result in a desire for a lesser amount of the other.

In some embodiments,

$$\vert \frac{{Z}_{40}}{{Z}_{20}} \vert$$

has a minimum value equal to one of 0.01, 0.02, 0.025, 0.03, 0.04 and 0.05, and a maximum value equal to one of 5.0, 4.0, 3.0, 2.0 and 0.7.

In some embodiments,

$$0.02 \leq \vert \frac{{Z}_{40}}{{Z}_{20}} \vert \leq 2.0$$

is preferable; and in other embodiments,

$$0.1 \leq \vert \frac{{Z}_{40}}{{Z}_{20}} \vert \leq 0.8$$

is preferable.

The term “0.01≦|Z₆₀≦1.0” indicates an absolute value of an amount of second order spherical aberration. Such a parameter provides a suitable amount of second order spherical aberration, which assists in distributing light energy more uniformly along the optical axis than is provided by the

$$\vert \frac{{Z}_{40}}{{Z}_{20}} \vert$$

term alone. It will be appreciated that an excessive amount of spherical aberration may be detrimental to contrast and/or acuity of the resultant image.

In some embodiments, |Z₆₀| has a minimum value (in waves) equal to one of 0.01, 0.02, 0.03, 0.04, 0.05 and 0.1, and a maximum value (in waves) equal to one of 1.0, 0.8, 0.6, 0.5, 0.4, 0.25 and 0.2.

In some embodiments, 0.01≦|Z₆₀|≦1.0. In some embodiments, 0.01≦|Z₆₀|≦0.5 is preferable; and in other embodiments, 0.01≦|Z₆₀|≦0.25 is preferable. In other embodiments, 0.02≦|Z₆₀|≦0.5.

In some embodiments, third order spherical aberration (Z₈₀) is controlled in addition to Z₄₀ and Z₆₀. In such embodiments, 0.001≦|Z₈₀|≦0.10 waves may be introduced. A wavefront described by Z₈₀ has components similar to both Z₄₀ and Z₆₀. In some embodiments, Z₈₀ may be used to fine tune the distribution of light along an axis.

In some embodiments, Z₈₀ has a minimum value (in waves) equal to one of 0.001, 0.005, 0.01, 0.02, and a maximum value (in waves) equal to one of 0.1, 0.75, 0.05 and 0.03.

In some embodiments, 0.001≦|Z₈₀|≦0.1 is preferable. In other embodiments, 0.001≦|Z₈₀|≦0.05 is preferable; and in other embodiments, 0.001≦|Z₈₀|≦0.02 is preferable.

In some embodiments, the aberrations as set forth above are achieved for all object locations in a range of locations along an axis from infinity to 1.0 diopter (i.e., 1 meter) when the lens is applied to an average eye. In other embodiments, the aberrations as set forth above are achieved for all locations along an axis in a range of locations from infinity to 1.5 diopters (i.e., 67 cm). In still other embodiments, the spherical aberration as set forth above is achieved for all locations along an axis in a range of locations from infinity to 2.0 diopters (i.e., 50 cm).

Some embodiments of lenses have a depth of field of at least 0.50 diopter for a given pupil diameter (e.g., 3 mm, 4 mm and/or 5 mm diameter). Some embodiments may have a depth of field of at least 0.75 diopters or 1.00 diopters or at least 1.25 diopters or at least 1.50 diopters. Typically, the depth of field is less than 2.00 diopters. The depth of field is disposed about an object distance that provides a best focus on the retina (assumed to be an object at infinity). The far depth of field may occur at infinity or −0.25 diopters (i.e., corresponding to a hyperopic object location) or further from the eye.

Some embodiments of lens zones according to aspects of the present invention have clear aperture diameters ranging from 2-5 mm or 3-5 mm or any other suitable clear aperture diameters. For example, diameters may be 2 mm, 3 mm, 4 mm, 5 mm or 6 mm. Alternatively, the diameter may be at least 2 mm, at least 3 mm, at least 4 mm, at least 5 mm.

In some embodiment, for the lens's clear aperture, the lens achieves the relevant amounts of

$$\vert \frac{{Z}_{40}}{{Z}_{20}} \vert ,$$

Z₆₀, and Z₈₀. It will be appreciated that, for lenses with in the scope of aspects of this invention, for apertures less than 3 mm in diameter (e.g., 2 mm or less), the amounts of

$$\frac{{Z}_{40}}{{Z}_{20}} ,$$

Z₆₀, and Z₈₀ are relatively small compared to the relevant, larger diameters (e.g. 3 mm, 4 mm and 5 mm) because the light passing therethrough is too close to the paraxial regime to achieve significant spherical aberration; it will also be appreciated that, despite the low spherical aperture for the small apertures, enhancement of depth of field is typically achieved with light passing through the larger apertures of the lens.

In some embodiment, for each of a 3 mm diameter aperture, 4 mm diameter aperture, and a 5 mm diameter aperture, a lens achieves the relevant amounts of

$$\frac{{Z}_{40}}{{Z}_{20}} ,$$

Z₆₀ and Z₈₀. In some embodiment, for each of a 3 mm aperture and a 4 mm aperture, a lens achieves the relevant amounts of

$$\frac{{Z}_{40}}{{Z}_{20}} ,$$

Z₆₀ and Z₈₀. In some embodiment, for at least one of a 3 mm aperture, 4 mm aperture, and a 5 mm aperture, a lens achieves the relevant amounts of

$$\frac{{Z}_{40}}{{Z}_{20}} ,$$

### Z₆₀ and Z₈₀.

Although in some embodiments the lens zone is circular, in other embodiments, the zone may have another suitable shape (e.g., square, annular, polygonal) (the maximum dimensions of said other shapes is referred herein as a diameter). A zone may have an area of at least 3 mm² or at least 7 mm² or at least 12 mm² or at least 18 mm² or at least 27 mm².

In some embodiments, for a lens applied to an average eye, the boundary of the clear aperture of a lens is determined by edges of the region of a lens that is optically corrected (i.e., the clear aperture is the image forming portion of the lens). In other embodiments, the boundary of the clear aperture is determined by a natural or implanted feature of the eye in which the lens is placed (e.g., an iris or implanted ring). For example, the maximum diameter of a wearer's iris can be 5 mm. It will also be appreciated that, if a feature of the eye will determine the clear aperture, the diameter of the clear aperture is at least partially determined by the location in the eye in which the lens is located (e.g., for a given feature, a clear aperture of a contact lens adapted to be located on a cornea will have a different clear aperture than the clear aperture of an IOL adapted to be located in a posterior chamber of the eye).

In some embodiments, an ophthalmic lens is adapted to provide suitable depth of field characteristics as set forth above after the lens is applied to the eye, the maximum clear aperture being 5 mm. In other embodiments, the ophthalmic lens is adapted to provide suitable depth of field characteristics as set forth above after the lens is applied to the eye, the lens having a maximum clear aperture of 4 mm; in yet other embodiments, the ophthalmic lens is adapted to provide suitable depth of field characteristics as set forth above after the lens is applied to the eye, the lens having a maximum clear aperture of 3 mm.

In some embodiments of the present invention, to achieve suitable performance characteristics, a lens includes at least one aspheric surface whose sag is defined by a conic term

$${z}_{standard} ( r ) = \frac{{cr}^{2}}{1 + \sqrt{1 - ( 1 + k ) {c}^{2} {r}^{2}}}$$

where c is the curvature of said surface, k the conic constant, and r a radial coordinate from the surface vertex. If k=0, then the surface would be spherical.

In some embodiments, the surface is described by a sag as follows:

z(r)=z_{s tan dard}(r)+α₁r²+α₂r⁴+α₃r⁶+α₄r⁸+α₅r¹⁰+α₆r¹²+ . . .

It will be appreciated that the equation includes a conic term and even-powered polynomial terms. According to aspects of the present invention at least one of the αₙ terms is non-zero. It will be understood that it is typically desirable that the number and magnitude of an terms selected to be non-zero be the minimum necessary to achieve a selected performance. By so controlling the number and magnitude of said terms, sensitivity to decentration can be reduced, and manufacturability and testing of lenses can be simplified.

It will also be appreciated that, to achieve a desired result, some embodiments of the present invention include even-powered polynomial components (as presented above). Such embodiments, typically, are capable of providing performance beyond lenses having surfaces with only a standard conic asphere (Z_{standard}). In some embodiments, the lenses include surfaces having only even-powered aspheric terms. It is further to be appreciated that although even-powered polynomial terms may be all that is necessary to achieve selected aberration performance for a lens, for some embodiments, odd-powered polynomial terms may be added. For example, odd-powered aspheric terms may be appropriately used with contact lens embodiments, where decentration is likely.

An optic having an enhanced depth of field according to aspects of the present invention may be used in an accommodative lens where, in addition to the depth of field achieved by accommodative movement, the depth of field of the lens, when stationary, is enhanced. For example, an optic according to aspects of the present invention can be used in a dual element accommodative lens as described in U.S. Pat. No. 6,488,708 issued Dec. 4, 2002, to Sarfarazi, or a single element accommodative lens as described in U.S. Pat. No. 5,674,282, issued Sep. 7, 1997, to Cumming.

Use of lenses as described above may be achieved using any suitable application technique. For example, a contact lens will be applied to a wearer's eye by placing the lens on the wearer's cornea; and an IOL will be applied to a wearer's eye by inserting the lens into a wearer's eye (e.g., into the wearer's eye in the posterior chamber or anterior chamber).

One example of a technique for designing lenses according to aspects of the present invention is through the use of optical design software. It will be appreciated that such software may be used to provide a lens prescription by specifying a merit function and using an iterative optimization technique.

One example of a merit function (in Zemax design software) that is suitable for designing lenses according to aspects of the present invention specifies three configurations of an optical system, the optical system including an ophthalmic lens applied to an average eye. The three configurations specify an object point at a near object distance (e.g., 1 meter) (the first configuration), an object point at an intermediate object distance (e.g., 5 meters) (the second configuration), and an object point at a far object distance (e.g., 10 meters)(the third configuration), respectively. For each of the first configuration and the second configuration, the merit function specifies that the target wavefront is flat at the image plane and that at least 10% of the energy is incident at a common image point in an image plane (e.g., on the retina). For the third configuration, the merit function specifies a target ratio within the range

$$0.01 \leq \vert \frac{{Z}_{40}}{{Z}_{20}} \vert \leq 5.0 ,$$

and a target amount of secondary spherical aberration within the range 0.01≦Z₆₀|≦1.0 waves of 550 nm light, as well as a wavefront that is flat at the image plane and that at least 10% of the energy is incident at the common image point. In some instances, it has been found appropriate to use Lagrange multipliers to increase the weighting on the

$$\vert \frac{{Z}_{40}}{{Z}_{20}} \vert$$

the and |Z₆₀| target values. It will be appreciated that |Z₈₀| within the appropriate range may be added. A focal length is also specified. A surface sag z(r) as set forth above may used, with radius r, conic constant k and even aspheric terms α₁, α₂, α₃, and α₄ used as variables. For example, the surface may be a surface of a single element ophthalmic lens or a surface of a multi-element ophthalmic lens. One or more surfaces of a lens may have parameters that are variable. An optimization may be performed, for example, for light at 550 nm or for the photopic spectrum.

In some instances, the first merit function is allowed to converge for a selected time to achieve a first surface optimization. The output of first optimization is then optimized a second time using a second merit function that comprises more than three configurations (e.g., about twenty configuration), the configurations specify more than four object locations between the near and far object location specified in the first optimization. Additionally, in some instances more than one aperture is specified (e.g. three aperture sizes of the lens may be specified for each object location). For each configuration, the merit function specifies that the target wavefront is flat at the image plane and that at least 10% of the energy is incident at the common image point. In some instances, in addition to the additional configurations, it is advantageous that the merit function specify a target geometric performance parameter be equal to zero (e.g., geometric modulation transfer function (MTF) is specified to be equal to zero) for object locations that are nearer than the near object distance and further than the far object distance (possibly including hyperopic image locations) thereby reducing the image quality outside of the desired range of object locations.

Examples of lens prescriptions providing suitable performance characteristics according to aspects of the present invention are provided below. The embodiments were designed using Zemax version Jan. 22, 2007. Zemax design software is available from Zemax Development Corporation of Bellevue, Wash.

Depths of field of the lenses can be calculated using the following pattern matching technique. A letter “E” (referred to as the “object E”) was input into an eye optical system that includes an average eye (i.e., the Liou-Brennan eye) with the subject lens suitably positioned therein. The “E” input was established using the Zemax Geometric Image Analysis Settings such that a 20/20, geometric convolved, output “E” (an “Output ‘E’”) was obtained with the following parameters—

| | |
|---|---|
| Field size - 0.083330 | Show - Grey Scale |
| Image size - 0.050000 | Source - Uniform |
| Rays - 50,000,000 | Total Watts - 1.0000 |
| Wavelengths - All | Parity - Even |
| Field - 1 | Configuration - Current |
| No. of Pixels - 400 | Reference- Primary Chief |
| Surface - Image | Remove Vignetting - On |

Depth of field is measured by first (e.g., prior to inputting an “E”) determining a retinal position corresponding to a best focus for the eye optical system (including the subject lens) for an object position of infinity with a 3 mm pupil diameter. It will be understood that best focus is determined using conventional techniques as the location where wavefront error is minimized. Output images are obtained with the object E disposed at various locations along an axis of the lens (e.g., along the optical axis of the lens) in object field of the lens. The output images (obtained using the traced rays) were saved as JPEG files.

Vision Assistant version 8.5 by National Instrument of Austin, Tex. was used to compare a perfect “E” (i.e., an “E” template having perfect contrast, consisting of only “1's” and “0's”) with each Output “E” using pattern matching. The comparisons were performed at various on-axis (along the optical axis) locations about the best focus location. It will be understood that, for a JPEG image used in Vision Assistant, the HSI—Intensity Plane is extracted. The comparisons were performed for object location at quarter diopter (0.25 D) increments about an object located at infinity. Accordingly, depths of field were determined in 0.25 diopter increments; however any suitable level of precision may be used.

A given depth of field was taken as including all object locations where a pattern matching score of at least 350 out of a 1000 was attained (where 350 is taken as the lowest value at which the “E” is discernable; and where 1000 is a perfect score). It will be appreciated that a depth of field can be calculated for a specific pupil diameter (e.g., 3 mm, 4 mm, 5 mm) as established in Zemax when defining the pupil size of the average eye.

### EXAMPLE 1

Table 2 is a prescription for a first example of an embodiment of a lens according to aspects of the present invention. Table 2 illustrates an example of a single-element, intraocular lens (IOL) made of an example hydrophilic acrylic material having an index of refraction equal to approximately 1.46 for light having a wavelengths of 0.546 micrometers; and as a function of wavelength (λ), n equals

$$1.38529196 + ( \frac{1.12901134 E - 02}{λ} ) + ( \frac{2.29091649 E - 04}{{λ}^{3.5}} ) ,$$

where wavelength is given in microns.

TABLE 2

| | Radius | Conic | Thickness | | | | |
|---|---|---|---|---|---|---|---|
| Surface | R (mm) | Constant k | (mm) | α₁ (mm) | α₂ (mm) | α₃ (mm) | α₄ (mm) |
| 1 | 46.911460 | −586.438853 | 0.705991 | 0.075437 | −5.9677E−03 | 2.447E−04 | 1.8332E−05 |
| 2 | −106.2958 | −1.6536E+06 | — | — | — | — | — |

The lens described in Table 2 is a 20 diopter lens having two aspheric surfaces. The first surface includes only even-powered aspheric terms (in addition to a conic term). To insert the IOL into the average eye model above, (1) the Anterior Surface, the Intermediate Surface and the Posterior Surface of the average eye are omitted; and (2) the surface 1 of the IOL is positioned 1.31 mm behind the iris.

Zernike coefficients for a wavefront at the retina (i.e., after the light passes through the lens) for an object position of infinity, a 1.0 diopter object position and a 1.5 diopter position are shown in Table 8 below. Zernike coefficients are shown for each of three pupil diameter sizes (3 mm, 4, mm and 5 mm) at each object position.

The depths of field (DOFs) measured along the optical axis of the lens using the pattern matching technique described above are as follows:

With a 3 mm pupil diameter, −0.25 diopters to 1.00 diopters (1.25 DOF)

With a 4 mm pupil diameter, −0.25 diopters to 1.25 diopters (1.50 DOF)

With a 5 mm pupil diameter, −0.25 diopters to 1.00 diopters (1.25 DOF)

### EXAMPLE 2

Table 3 is a prescription for a second example of an embodiment of a lens according to aspects of the present invention. Table 3 illustrates an example of a single-element, intraocular lens (IOL) made of an example hydrophilic acrylic material having an index of refraction as set forth above in Example 1.

TABLE 3

| | Radius | Conic | Thickness | | | | |
|---|---|---|---|---|---|---|---|
| Surface | R (mm) | Constant k | (mm) | α₁ (mm) | α₂ (mm) | α₃ (mm) | α₄ (mm) |
| 1 | −7.119639 | −5.710828 | 0.730965 | 0.082504 | −9.1686E−03 | 1.6947E−03 | −1.537E−04 |
| 2 | −7.277217 | 2.592710 | — | — | — | — | — |

The lens described in Table 3 is a 20 diopter lens having two aspheric surfaces. The first surface includes only even-powered aspheric terms (in addition to a conic term).

Zernike coefficients for a wavefront at the retina (i.e., after the light passes through the lens) for an object position of infinity, a 1.0 diopter object position and a 1.5 diopter position are shown in Table 8 below. Zernike coefficients are shown for each of three pupil diameter sizes (3 mm, 4, mm and 5 mm) at each object position.

The depths of field (DOFs) measured along the optical axis of the lens using the pattern matching technique described above are as follows:

With a 3 mm pupil diameter, infinity (0 diopters) to 1.50 diopters (1.50 DOF)

With a 4 mm pupil diameter, infinity (0 diopters) to 1.50 diopters (1.50 DOF)

With a 5 mm pupil diameter, −0.25 diopters to 1.00 diopters (1.25 DOF)

### EXAMPLE 3

Table 4 is a prescription for an example of a single-element, contact lens made of a Hydrogel material having an index of refraction (n) equal to approximately 1.41 for the d-wavelength of 0.589 micrometers; and as a function of wavelength (λ), n equals

$$1.43892094 + ( \frac{1.10429710 E - 02}{λ} ) + ( \frac{2.49170954 E - 04}{{λ}^{3.5}} ) ,$$

where wavelength is given in microns.

TABLE 4

| | Radius | Conic | Thickness | | | | | |
|---|---|---|---|---|---|---|---|---|
| Surface | R (mm) | Constant k | (mm) | α₁ (mm) | α₂ (mm) | α₃ (mm) | α₄ (mm) | α₅ (mm) |
| 1 | 8.077 | −0.951 | 0.160 | −2.519E−03 | 1.971E−03 | −1.444E−04 | 5.096E−06 | −6.507E−08 |
| 2 | 8.6 | 0 | — | — | — | — | — | — |

The lens described in Table 4 is a +3 diopter lens having one aspheric surface. The first surface includes only even-powered aspheric terms (in addition to a conic term).

To apply the lens of Table 4 to the average eye, Surface 2 is located with zero separation from the Anterior Cornea surface (i.e., in contact with the Anterior Cornea Surface).

Zernike coefficients for a wavefront at the retina (i.e., after the light passes through the lens) for an object positioned at infinity, a 1.0 diopter object position and a 1.5 diopter position are shown in Table 8 below. Zernike coefficients are shown for each of three pupil diameter sizes (3 mm, 4, mm and 5 mm) at each object position.

### EXAMPLE 4

Table 5 is a prescription for a fourth example of an embodiment of a lens according to aspects of the present invention. Table 5 illustrates another example of a single-element, intraocular lens (IOL) made of an example hydrophilic acrylic having an index of refraction as set forth above in Example 1.

TABLE 5

| | Radius | Conic | Thickness | | | | |
|---|---|---|---|---|---|---|---|
| Surface | R (mm) | Constant k | (mm) | α₁ (mm) | α₂ (mm) | α₃ (mm) | α₄ (mm) |
| 1 | 19.70089 | 57.266461 | 1.000 | 0.044877 | −4.083E−03 | 2.3631E−04 | −1.760E−05 |
| 2 | −47.55734 | −1.0209E+39 | — | — | — | — | — |

Zemike coefficients for a wavefront at the retina (i.e., after the light passes through the lens) for an infinite object position, a 1.0 diopter object position and a 1.5 diopter position are shown in Table 8 below. Zemike coefficients are shown for each of three pupil diameter sizes (3 mm, 4, mm and 5 mm) at each object position.

The depths of field (DOFs) measured along the optical axis of the lens using the pattern matching technique described above are as follows:

With a 3 mm pupil diameter, infinity (0 diopters) to 1.00 diopters (1.00 DOF)

With a 4 mm pupil diameter, infinity (0 diopters) to 1.00 diopters (1.00 DOF)

With a 5 mm pupil diameter, infinity (0 diopters) to 1.00 diopters (1.00 DOF)

### EXAMPLE 5

Table 6 is a prescription for a fifth example of an embodiment of a lens according to aspects of the present invention. Table 6 illustrates an example of a dual-element, intraocular lens (IOL) made of an example hydrophilic acrylic having an index of refraction as set forth above in Example 1. The first surface and the fourth surface include only even-powered aspheric terms (in addition to a conic term).

TABLE 6

| | Radius | Conic | Thickness | | | | |
|---|---|---|---|---|---|---|---|
| Surface | R (mm) | Constant k | (mm) | α₁ (mm) | α₂ (mm) | α₃ (mm) | α₄ (mm) |
| 1 | 3.791805 | −0.047570 | 1.485996 | −2.272E−03 | 1.830E−03 | 1.479E−06 | −1.57E−04 |
| 2 | −6.892086 | 1.050200 | — | — | — | — | — |
| 3 | −4.464113 | −1.222105 | 0.200 | — | — | — | — |
| 4 | −103.855 | −2.668026 | — | 0.017978 | 3.761E−03 | −1.214E−05 | −2.728E−04 |

The lens described in Table 6 is a 20 diopter lens having four aspheric surfaces.

Similar to a single element IOL, to insert the dual element IOL of Table 5 into the average eye model above, (1) the Anterior Surface, the Intermediate Surface and the Posterior Surface of the average eye are omitted; and (2) Surface 1 of the IOL is positioned 1.31 mm behind the iris.

Zemike coefficients for a wavefront at the retina (i.e., after the light passes through the lens) for an object position of infinity, a 1.0 diopter object position and a 1.5 diopter position are shown in Table 8 below. Zernike coefficients are shown for each of three pupil diameter sizes (3 mm, 4, mm and 5 mm) at each object position.

The depths of field (DOFs) measured along the optical axis of the lens (while the lens remains stationary (i.e., it is assumed that there is no accommodative movement)) using the pattern matching technique described above are as follows:

With a 3 mm pupil diameter, −0.25 diopters to 0.50 diopters (0.75 DOF)

With a 4 mm pupil diameter, −0.25 diopters to 0.75 diopters (1.00 DOF)

With a 5 mm pupil diameter, −0.25 diopters to 0.75 diopters (1.00 DOF)

### EXAMPLE 6

Table 7 is a prescription for an example of a single-element, contact lens made of a silicone hydrogel material having an index of refraction (n) equal to approximately 1.417 for the d-wavelength of 0.589 micrometers; and as a function of wavelength (λ), n equals

$$equals 1.38059501 + ( \frac{2.19201339 E - 02}{λ} ) + ( \frac{- 8.76677677 E - 05}{{λ}^{3.5}} ) ,$$

where wavelength is given in microns.

TABLE 7

| | Radius | Conic | Thickness | | | | |
|---|---|---|---|---|---|---|---|
| Surface | R (mm) | Constant k | (mm) | α₁ (mm) | α₂ (mm) | α₃ (mm) | α₄ (mm) |
| 1 | 8.57 | — | 0.070 | — | −1.20e−3 | 1.45e−4 | — |
| 2 | 8.20 | — | — | — | — | — | — |

The lens described in Table 7 is a −3 diopter lens having one aspheric surface. The first surface includes only even-powered aspheric terms (in addition to a conic term).

To apply the lens of Table 7 to the average eye, Surface 2 is located with zero separation from the Anterior Cornea surface (i.e., in contact with the Anterior Cornea Surface).

Zernike coefficients for a wavefront at the retina (i.e., after the light passes through the lens) for an infinite object position, a 1.0 diopter object position and a 1.5 diopter position are shown in Table 8 below. Zernike coefficients are shown for each of three pupil diameter sizes (3 mm, 4, mm and 5 mm) at each object position.

The depths of field (DOFs) measured along the optical axis of the lens using the pattern matching technique described above are as follows:

With a 3 mm pupil diameter, infinity (0 diopters) to 1.25 diopters (1.25 DOF)

With a 4 mm pupil diameter, infinity (0 diopters) to 1.25 diopters (1.25 DOF)

With a 5 mm pupil diameter, infinity (0 diopters) to 1.25 diopters (1.25 DOF)

| | | Infinity | | | +1.0 D | | | +1.5 D | | |
|---|---|---|---|---|---|---|---|---|---|---|
| Lens | Pupil | Z40/Z20 | Z60 | Z80 | Z40/Z20 | Z60 | Z80 | Z40/Z20 | Z60 | Z80 |
| Ex. 1 | 3 | 0.289 | 0.019 | 0.001 | 0.180 | 0.019 | 0.001 | 0.152 | 0.019 | 0.001 |
| | 4 | 0.237 | 0.092 | 0.002 | 0.175 | 0.092 | 0.002 | 0.155 | 0.092 | 0.002 |
| | 5 | 0.179 | 0.361 | 0.004 | 0.142 | 0.360 | 0.004 | 0.129 | 0.360 | 0.004 |
| Ex. 2 | 3 | −0.759 | 0.038 | 0.000 | 0.127 | 0.038 | 0.000 | 0.080 | 0.038 | 0.000 |
| | 4 | 0.362 | 0.124 | −0.000 | −0.122 | 0.124 | −0.000 | −0.073 | 0.124 | −0.000 |
| | 5 | 0.352 | −0.025 | −0.005 | −0.524 | −0.025 | −0.005 | −0.234 | −0.025 | −0.005 |
| Ex. 3 | 3 | 0.256 | −0.043 | 0.000 | 0.381 | −0.042 | 0.000 | 0.504 | −0.042 | 0.000 |
| | 4 | 0.204 | −0.207 | 0.001 | 0.259 | −0.204 | 0.001 | 0.298 | −0.202 | 0.001 |
| | 5 | 0.149 | −0.590 | 0.000 | 0.181 | −0.583 | 0.000 | 0.201 | −0.579 | 0.000 |
| Ex. 4 | 3 | 0.408 | 0.010 | 0.000 | 0.116 | 0.010 | 0.000 | 0.085 | 0.010 | 0.000 |
| | 4 | 0.245 | 0.057 | 0.001 | 0.103 | 0.057 | 0.001 | 0.080 | 0.057 | 0.001 |
| | 5 | −0.023 | 0.323 | 0.022 | −0.010 | 0.323 | 0.022 | −0.008 | 0.323 | 0.022 |
| Ex. 5 | 3 | 0.818 | −0.005 | 0.000 | −0.086 | −0.005 | 0.000 | −0.056 | −0.005 | 0.000 |
| | 4 | 0.368 | −0.016 | 0.002 | −0.136 | −0.015 | 0.002 | −0.083 | −0.015 | 0.002 |
| | 5 | 0.463 | 0.194 | 0.029 | −0.776 | 0.198 | 0.030 | −0.342 | 0.200 | 0.031 |
| Ex. 6 | 3 | −2.376 | 0.050 | 0.000 | 0.366 | 0.050 | 0.000 | 0.233 | 0.050 | 0.000 |
| | 4 | 0.681 | 0.275 | −0.001 | 0.025 | 0.275 | −0.001 | 0.017 | 0.275 | −0.001 |
| | 5 | 0.765 | 0.906 | −0.010 | 4.538 | 0.910 | −0.009 | −3.155 | 0.912 | −0.009 |

Selection of the power of a lens to be applied to a wearer's eye may be achieved using any suitable technique. According to one conventional technique, the power of the lens is selected to place the wearer's maximum contrast at infinity when the wearer's eye muscles are relaxed (i.e., an eye is made to be emmotropic). It will be appreciated that by so selecting a lens, lenses according to aspects of the present lens provide a depth of field about the wearer's infinite object location. It will be further appreciated that the effect of such a depth of field is to provide a margin of error such that, if the lens and/or eye do not operate as expected, a wearer's vision is not compromised.

As one of ordinary skill in the art would understand, with conventional pre-operative testing, it may be difficult to select a lens that, after surgical implantation, will precisely provide maximum contrast at infinity. For example, the lack of precision may be due to imprecise measurement of the eye, imprecise location of the lens within the eye due to the unpredictable mechanical characteristics of the eye tissue, and/or the unpredictable nature of post-operative healing of eye tissue. Accordingly, while in the cases of conventional lenses having conventional depths of field, the lenses may not make a wearer emmetropic (i.e., a wearer may not focus light from an object at infinity onto his/her retina when the eye muscles are relaxed), lenses according aspects of the present invention provide a margin of error arising from the extended depth of field which will ensure that the wearer is emmetropic.

According to an alternative lens selection technique, a lens is selected such that optimal vision occurs at the wearer's hyperfocal distance (i.e., the distance that results in the maximum allowable defocus of objects located at infinity such that suitable contrast occurs on the retina for objects at infinity). According to such techniques, a portion of the lens's depth of field extends from the hyperfocal distance closer to the eye. By so selecting the power of lenses, lenses according to aspects of the present invention (i.e., lenses having enhanced depths of field) provide a substantial distance over which a wearer will have suitable vision.

It will be appreciated that the above lenses have been designed to achieve a particular performance in an average eye. Performance of a particular wearer's eye with the lens applied thereto may vary. According to aspects of the present invention, a combined procedure is performed on an eye. For example, according to aspect of the present invention a lens is applied to an eye (e.g., implanted in the eye) and a refractive surgery is performed on the eye such that the combination adjust

$$\vert \frac{{Z}_{40}}{{Z}_{20}} \vert ,$$

|Z₆₀| and, possibly, |Z₈₀| to achieve a wavefront at the retina having qualities as described above. Application of the lens and the refractive procedure may be performed in any order. In some instances the lens is applied to the eye before the refractive surgery is performed. It will be appreciated that a wavefront at the retina can be determined for example using an ophthalmic aberrometer.

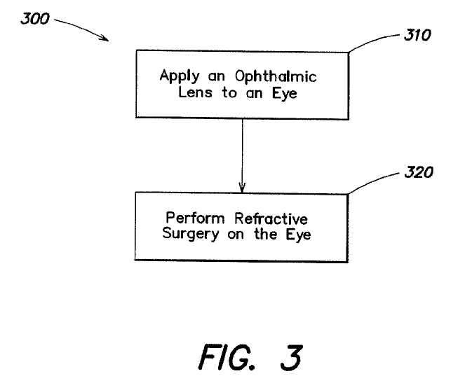

FIG. 3 is a flow chart illustrating a method 300 according to an aspect of the invention. In a first step (310) an ophthalmic lens (e.g., an IOL) is implanted in an eye. In a second step, refractive surgery is performed such that, for all object locations along an axis in a range from infinity to 1.0 diopters, for light having a wavelength 550 nm, the wavefront at the retina formed by the IOL has the following characteristics

$$0.01 \leq \vert \frac{{Z}_{40}}{{Z}_{20}} \vert \leq 5.0 , and$$

0.01≦|Z₆₀|≦1.0 waves of 550 nm light. In alternative embodiments, amount or range of amounts of

$$\vert \frac{{Z}_{40}}{{Z}_{20}} \vert ,$$

|Z₆₀|, and |Z₈₀| as described herein may be achieved.

For example, the refractive surgery of the second step may comprise performing a laser ablation surgery (e.g, a LASIK procedure), a laser relaxation surgery (e.g., a femptosecond procedure), or applying a corneal inlay (that includes or does not include a lens) or applying a corneal onlay (that includes or does not include a lens).

The refractive surgery step may comprise increasing or decreasing one or more aberrations of the eye.

The refractive surgery may comprise adding a tilt component to the cornea of the eye (e.g., a prismatic effect) to align an optical axis of the cornea of the eye with the optical axis of the IOL. In some embodiments, adding tilt may be used to alter higher-order aberrations of the eye optical systems. For example, an amount of coma in the eye optical system

It will be appreciated that, although the above methods and procedures are related to ophthalmic lenses for providing eye optical systems with extended depths of field, any suitable ophthalmic procedure may be used to achieve wavefronts at a retina such that, for all object locations along an axis in a range from infinity to 1.0 diopter, for light having a wavelength 550 nm, the wavefront at the retina formed by the IOL has the following characteristics

$$0.01 \leq \vert \frac{{Z}_{40}}{{Z}_{20}} \vert \leq 5.0 , and$$

0.01≦|Z₆₀|≦1.0 waves of 550 nm light. In alternative embodiments, amount or range of amounts of

$$\vert \frac{{Z}_{40}}{{Z}_{20}} \vert$$

|Z₆₀|, and |Z₈₀| as described herein may be achieved.

In some instances, non-lens ophthalmic apparatus (e.g., a corneal inlay or onlay that does not include a lens) are used alone or in combination with an ophthalmic lens. In some instances, a non-implant refractive surgical technique (e.g. LASIK, keratotemy, keratectomy) may be used without an ophthalmic apparatus applied to the eye (e.g., neither an ophthalmic lens nor a non-lens corneal inlay or non-lens corneal onlay is used) to achieve an amount or range of amounts of

$$\vert \frac{{Z}_{40}}{{Z}_{20}} \vert ,$$

|Z₆₀|, and |Z₈₀| as described herein.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An ophthalmic lens, comprising:
at least one optic comprising a lens zone, the zone configured such that, when the lens is located in an average eye, for all object locations along an axis in a range from infinity to 1.0 diopters, for light having a wavelength 550 nm, the wavefront at the retina formed by the lens zone has the following characteristics
$$0.01 \leq \vert \frac{{Z}_{40}}{{Z}_{20}} \vert \leq 5.0 , and$$
0.01≦|Z₆₀|≦1.0 waves of 550 nm light.

2. The lens of claim 1, wherein the lens has a depth of field of at least 1.0 diopter.

3. The lens of claim 1, wherein the wavefront at the retina formed by the zone has 0.001≦|Z₈₀|≦0.1 waves at all object locations between infinity and 1.0 diopter for light having a wavelength 550 mn.

4. The lens of claim 1, wherein
$$\vert \frac{{Z}_{40}}{{Z}_{20}} \vert \leq 3.0 .$$

5. The lens of claim 1, wherein
$$0.25 \leq \vert \frac{{Z}_{40}}{{Z}_{20}} \vert \leq 0.8 .$$

6. The lens of claim 1, wherein |Z₆₀|≦0.5 waves.

7. The lens of claim 1, wherein |Z₆₀|≦0.25 waves.

8. The lens of claim 1, wherein |Z₈₀|≦0.075 waves.

9. The lens of claim 1, wherein |Z₈₀|≦0.05 waves.

10. The lens of any of claim 1, wherein the specified values of
$$\vert \frac{{Z}_{40}}{{Z}_{20}} \vert$$
and |Z₆₀| and are achieved at all object locations between infinity and 1.5 diopters.

11. The lens of any of claim 3, wherein the specified values of
$$\vert \frac{{Z}_{40}}{{Z}_{20}} \vert ,$$

|Z₆₀| and |Z₈₀| are achieved at all object locations between infinity and 1.5 diopters.

12. The lens of any of claim 3, wherein the specified values of
$$\vert \frac{{Z}_{40}}{{Z}_{20}} \vert ,$$

|Z₆₀| and |Z₈₀| are achieved at all object locations between infinity and 2.0 diopters.

13. The lens of claim 1, wherein the lens zone has a diameter of at least 3.0 mm.

14. The lens of claim 1, wherein the lens zone has a diameter of at least 4.0 mm.

15. The lens of claim 1, wherein the lens zone has a diameter of at least 5.0 mm.

16. The lens of claim 1, wherein the lens comprises only even-powered aspheric terms.

17. The lens of claim 1, wherein the lens is an accommodative lens.

18. The lens of claim 1, wherein the lens is a dual element accommodative lens.

19. The lens of claim 1, wherein the clear aperture is determined by the region of the optic that is optically corrected.

20. The lens of claim 1, wherein the lens zone is the only zone of the lens.

21. The lens of claim 1, wherein the lens is a multifocal lens, and the lens zone is one of a plurality of lens zones.

22. The lens of claim 1, wherein the lens has only two lens zones.

23. The lens of claim 1, wherein the lens zone has an area of at least 3 mm².

24. The lens of claim 1, wherein the lens zone has an area of at least 7 mm².

25. The lens of claim 1, wherein the lens zone has an area of at least 12 mm².

26. The lens of claim 1, wherein
$$\vert \frac{{Z}_{40}}{{Z}_{20}} \vert \geq 0.02 .$$

27. The lens of claim 1, wherein
$$\vert \frac{{Z}_{40}}{{Z}_{20}} \vert \geq 0.03 .$$

28. The lens of claim 1, wherein Z₆₀≧0.02.

29. The lens of claim 1, wherein Z₆₀≧0.03.

30. The lens of claim 1, wherein Z₈₀≧0.005.

31. The lens of claim 1, wherein Z₈₀≧0.01.

32. A method comprising:
performing an ophthalmic procedure on an eye optical system such that, for all object locations along an axis in a range from infinity to 1.0 diopters, for light having a wavelength 550 nm, the wavefront at the retina formed by the eye optical system has the following characteristics
$$0.01 \leq \vert \frac{{Z}_{40}}{{Z}_{20}} \vert \leq 5.0 , and$$
0.01≦|Z₆₀|≦1.0 waves of 550 nm light.

33. The method of claim 32, wherein the step of performing an ophthalmic procedure comprises applying an ophthalmic lens to the eye optical system.

34. The method of claim 33, wherein the ophthalmic lens is an intraocular lens, and the step of applying the lens comprises inserting the lens into the eye.

35. The method of claim 34, wherein the step of performing an ophthalmic procedure is a combined procedure, the procedure further comprising performing refractive surgery on the eye.

36. The method of claim 35, wherein the refractive surgery is performed after the intraocular lens is applied.

37. The method of claim 35, wherein the step of performing refractive surgery comprises performing a laser refractive procedure on the eye.

38. The method of claim 36, wherein the step of performing refractive surgery comprises altering a tilt component to the cornea.

39. The method of claim 35, wherein the step of performing refractive surgery comprises adding a non-lens corneal inlay or a non-lens corneal onlay to the eye.
